(19) United States
(12) Patent Application Publication
(10) Pub. No.: US 2013/0323321 A1
(43) Pub. Date: Dec. 5, 2013

(54) PERSONAL HYGIENE COMPOSITION, ARTICLE OF MANUFACTURE AND METHOD COMPRISING CHAKRA STONE

(71) Applicant: Renan Garcia, Miami, FL (US)

(72) Inventor: Renan Garcia, Miami, FL (US)

(21) Appl. No.: 13/487,790

(22) Filed: Jun. 4, 2012

(51) Int. Cl. A61K 8/19; B65D 71/00; A61Q 5/02; A61K 33/00; A61Q 19/10

(52) U.S. Cl. 424/600; 510/130; 510/119; 206/581; 206/232

(57) ABSTRACT

Chakra stones or crystals are included as part of a personal hygiene or body cleansing composition as a novelty item or for aesthetic or decorative purposes. The chakra stones or crystals can also be used for their spiritual significance and for realigning, revitalizing, or balancing unbalanced chakra energies of the body.

### BACKGROUND

According to certain traditions and beliefs, there are seven power points in the body, referred to as “chakras,” which circulate energy or the life force, also known as “prana.” The term “chakra” is from Sanskrit, meaning “wheel of light.” The seven chakras are vertically aligned in the center of the body, close to the spine. If the chakras are unbalanced, it is believed that one's physical, mental and/or spiritual health can be affected.

Each of the seven chakras is associated with a stone or crystal which can provide an energy or vibration or frequency which aligns the associated chakra. Thus, the frequency of each stone's energy is pulled through the associated chakra to realign, revitalize and replenish the energies of the body. The chakra stones are also understood to be associated with a particular color.

A personal hygiene composition, such as a soap, shampoo, body cleanser or moisturizer that contains one or more chakra stones can be useful as a novelty item or, to those that utilize the energies, vibrations or frequencies of the chakra stones, a beneficial composition for realigning an unbalanced chakra of the body.

Minerals, including crystals or gemstones, have been previously included in cleansing compositions. However, such minerals or crystals were typically provided to affect the physical property of the composition, itself. Nothing in the prior art is known which describes the association of chakras or balancing of energies using such crystals or minerals in a cleansing composition.

U.S. Pat. No. 7,294,340, to Sung, et al. describes nanoparticles of diamond included in a shampoo composition. However, the nanodiamond crystals are provided for their ability of to bond with biological materials and to improve mechanical strength of the composition.

U.S. Pat. No. 7,179,152 describes the use of micronized precious stones useful as an abrasive component in a composition or on an apparatus useful for microdermabrasion (e.g., microexfoliation, particle skin resurfacing or the like).

U.S. Pat. No. 6,036,965, to Gubernick, et al., describes cosmetic compositions for skin or hair cleansing which includes the borosilicate mineral, tourmaline. Tourmaline is stated to have a variety of unusual pyroelectric and piezoelectric properties. These properties, however, relate to physical or chemical properties imparted to the components of the composition and its function as a cleansing composition.

U.S. Pat. No. 4,857,306 describes a cosmetic composition comprising precious stones in powder form for decorative use, i.e., for application of the precious stone powder to areas of the body including the face, nails, or hair.

Nothing in the prior art teaches or suggests a cleansing composition to which macro-sized stones are added. Nor does the prior art teach or suggest the addition of stones used in association with chakras or the balancing of energies associated with a chakra.

### BRIEF SUMMARY OF THE INVENTION

The subject invention concerns a personal hygiene composition, such as a skin or hair cleanser, e.g., a soap or shampoo, a moisturizer or conditioner, or the like, which can comprise a chakra stone or crystal or a plurality of chakra stones or crystals within the composition. In a preferred embodiment, the composition is a substantially transparent or translucent liquid or gel composition in which the chakra stones are visible by the consumer of the composition. Thus, the chakra stones within the cleanser composition are provided having a size that is not in powder form, e.g., not ground, micronized or in nanoparticulate form. The size range of the chakra stones is on the order of at least millimeters in size, and preferably measuring one centimeter or greater.

The composition can comprise at least one chakra stone or crystal, and preferably comprises a plurality of chakra stones or crystals. A preferred embodiment of the invention comprises a personal hygiene composition, such as a cleansing composition, containing at least seven chakra stones or crystals, each respectively representing or associated with a chakra of the body.

The composition can be provided as an article of manufacture comprising the cleansing composition and chakra stone or crystal, wherein the composition comprising a chakra stone or crystal is provided within a container. Preferably, the container comprises, at least in part, a substantially clear or transparent or translucent material, e.g., a bottle or dispensing tube container, for containing the cleansing composition comprising a chakra stone or crystal. Written instruction relating to the cleansing composition, the chakra stone or crystal, use of the composition or chakra stone, or a combination thereof, can be include with or on the container.

A composition comprising one or more chakra stones, contained within or bounded by a bottle or tube container can therefore be an article of manufacture. Preferably, the article of manufacture of the invention comprises a container which is a bottle or dispensing tube bounding the composition and chakra stone, and further comprises an outer packaging which can independently comprise written instruction relating to the cleansing composition, the chakra stone or crystal, use of the composition or chakra stone, or a combination thereof.

The subject invention also includes one or more methods of using the composition or article of manufacture, including, conventional use of a cleansing, moisturizing or conditioning composition to clean an area of the body. The method of using the invention can further comprise the cleansing use of the cleansing composition which, concomitantly, can realign a chakra of the body associated with the particular chakra stone or crystal contained within the composition.

A further method of using the composition of the subject invention includes using the chakra stones or crystals after the cleansing composition has been expended or “used up.” For example, the chakra stones can be removed from the container and used in a traditional chakra realignment procedure or technique. Preferably, the composition used in this method comprises a plurality of chakra stones or crystals and, more preferably, comprises at least one chakra stone or crystal representing each of the seven major chakras of the body. The chakra stones can be removed from the bottle or tube, optionally rinsed, and placed or arranged to form a mandala.

The chakra stones can be a precious or semi-precious stone or gemstone, or a mineral, in the form of a substantially round or amorphous shape. The chakra stone can also be provided as a geometric shape or crystal form. Each stone or crystal can be chosen or selected for its color, which is associated with a particular energy or frequency which, in turn, is associated with a particular chakra of the body known or believed to be affected or balanced by that frequency or energy of the chakra stone or crystal.

The composition or article of manufacture of the subject invention can be provided as a novelty item or a decorative item in view of the aesthetically pleasing appearance of the chakra stone or stones visible within the composition, container or outer packaging. The subject invention can also be useful as a chakra balancing composition for persons that traditionally employ chakra stones to realign, revitalize and replenish unbalanced chakra energies of the body.

### BRIEF DESCRIPTION OF THE DRAWINGS

FIG. 1 shows an embodiment of an article of manufacture and composition according to the subject invention.

FIG. 2 shows an embodiment of an article of manufacture and composition according to the subject invention, illustrating written instruction included as part of or attached to, the container.

FIG. 3 shows an embodiment of an article of manufacture and composition according to the subject invention, illustrating an outer packaging housing a container containing the composition and chakra stone.

FIG. 4 illustrates a method of using chakra stones obtained from the composition of the subject invention to form a mandala arrangement.

### DETAILED DESCRIPTION

The subject invention concerns a personal hygiene composition comprising one or more chakra stones. The personal hygiene composition is referred to herein as a “cleanser composition,” and means any soap, shampoo, body cleanser, moisturizer (including moistening of the eye), conditioner, hair spray or cosmetic composition that can be applied to the body of an individual for its intended purpose, including to clean, refresh, moisturize, hydrate or replace desired components of the skin, hair, nails, or the like. The composition according to the subject invention can be provided in a container as an article of manufacture, and can further comprise an outer packaging.

The container, packaging, or article of manufacture comprising the container or packaging, can independently comprise written instruction or information relating to the composition, chakra stone, or uses thereof. The composition or article of manufacture of the invention can be useful as a novelty item or, to those that are familiar with or utilize the energies, vibrations or frequencies of the chakra stones, as a beneficial composition for realigning one or more unbalanced chakra of the body.

A composition according to the subject invention comprises, for example, a personal cleansing or moisturizing liquid or gel containing at least one chakra stone known to be associated with a chakra of the body. By a “personal cleansing liquid or gel” is meant a skin, nail, or hair soap, detergent, or treatment for application to the skin, hair, or nails to clean, moisturize or condition the skin, nail or hair. Personal cleansing liquids are well known in the art and are not critical to the invention so long as they are safe and effective. Preferably, the cleansing liquid comprises a soap or shampoo or other hair treatment (e.g., hairspray) formulation as is known in the art.

A soap or shampoo used in accordance with the subject invention is preferred to be in liquid or gel form because such compositions are known to be available as a clear formulation. However, a solid or semi-solid form can also be used. By a “clear” soap or shampoo formulation is meant a substantially transparent or translucent composition whereby the chakra stone or crystal is visible therein when added to the cleansing composition. “Clear” is not limited to “colorless” and can include a color, hue or tint so long as the composition is substantially transparent or translucent allowing visibility of the stone or crystal within the liquid or gel for its aesthetic appeal to a consumer.

A cleansing composition of the subject invention can comprise more than one chakra stone or crystal, and more preferably comprises at least seven chakra stones or crystals associated with the known seven chakras of the body.

As described herein, each chakra energy of the body is associated with one or more color of chakra stone or crystal. For example, a preferred composition can comprise seven chakra-balancing stones, such as: opal; amethyst; sodalite or blue agate; emerald, aventurine or rose quartz; citrine; carnelian, and red tiger eye. These chakra stones correspond, respectively, to the colors: white (Crown Chakra); violet or purple (Crown or Third Eye Chakra); blue (Throat Chakra); green or pink (Heart Chakra); yellow (Solar Plexus Chakra); orange (Sacral Chakra) and red (Root Chakra).

A composition that includes more than seven stones or crystals can comprise: Tiger Eye—First, Second and/or Third Chakras; Red Jasper—First Chakra; Hematite—Second Chakra; Orange Calcite—Third Chakra; Rose Quartz—Fourth Chakra; Aventurine—Fourth Chakra; sodalite—Fifth and/or Sixth Chakras; Amethyst—Seventh Chakra; and Clear Crystal Quartz—All Chakras.

The composition comprising a personal cleansing liquid or gel can be included in an article of manufacture which comprises the cleansing liquid or gel, one or more chakra stones or crystals, contained within a container, such as a bottle, dispensing tube, tub, jar, or canister comprising a wall or housing which is substantially clear in at least a portion of the wall or housing to allow visibility by a consumer of the chakra stone or crystal within the container. Preferably the container comprises a clear, transparent, or translucent glass or plastic material which provides visibility of the contents of the container for the entire shelf-life of the contents.

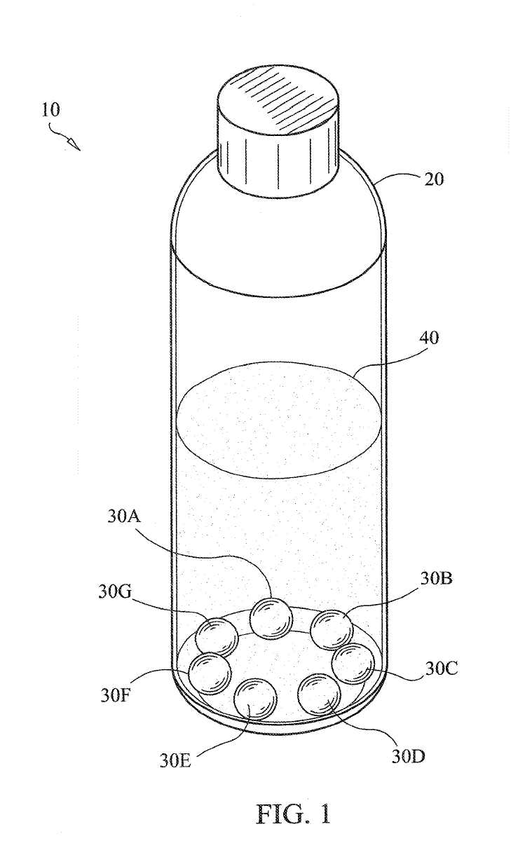

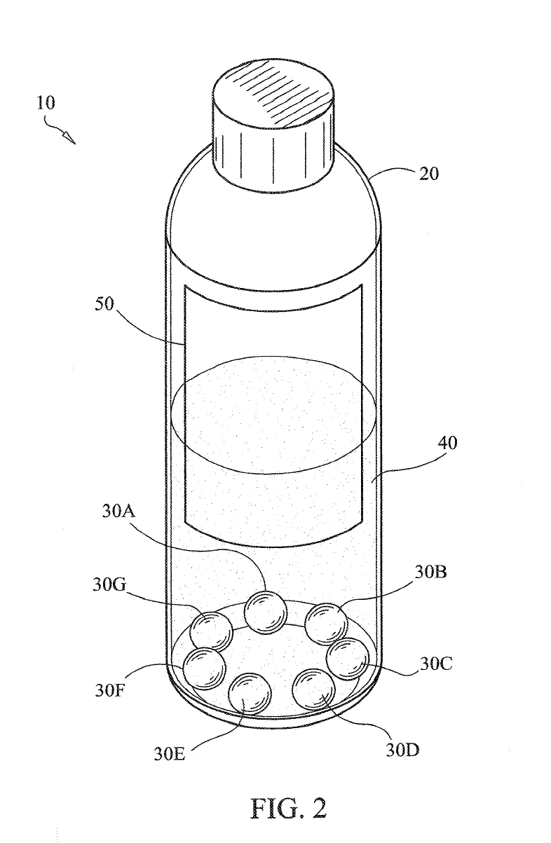

An example of a shampoo bottle according to the subject invention is illustrated in FIG. 1 and FIG. 2. Shown in FIG. 1 is an article of manufacture 10 comprising a container 20, which can contain one or more chakra stones within a liquid or gel cleansing composition 40. Shown here is an embodiment comprising seven chakra stones 30A-30G.

Chakra stones 30A-30G can be any color or combination of color selected from clear, white, violet, purple, blue, green, turquoise, pink, yellow, orange, red, black, brown, or grey. For example, 30A can be white; 30B can be purple; 30C and be blue; 30D can be green or pink; 30E can be yellow; 30F can be orange and 30G can be red or black.

As illustrated, container 20 and composition 40 are clear, transparent or translucent such that the chakra stones are visible therein.

The chakra stone or stones used in the article of manufacture of the subject invention are not limited by size or shape, so long as the stone or stone is recognized as a chakra stone or crystal by the naked eye. Therefore, a stone or crystal structure having a size greater than one millimeter, preferably on the order of tens of millimeters to centimeter size, or larger, can be used. For example, a typical stone or crystal used in connection with the subject invention is about 1-2 centimeters in diameter.

The size of the stone has no upper limit, but from a practical standpoint, has an upper size limit controlled by the size of the container. Therefore, the stones or crystals of the invention should easily and aesthetically “fit” within the container, preferably but not necessarily, providing that all of the stones fit within a single layer at the bottom of the container as shown in the Figures.

Another consideration for determining the size of the chakra stone or crystal concerns the manufacture of the article of the invention. The chakra stone or crystal should have a size that allows for easy and efficient placement of the chakra stone or crystal through the opening of the container, such as the neck of a bottle which can be size-limiting for placing contents therein.

The stones or crystals used for the subject invention are not ground or in the form of a powder, and are not micronized or provided in micro- or nano-particulate (or smaller) form.

The shape of the chakra stone or crystal can also vary and are not limiting of the invention. Stones and crystals having a variety of natural shapes can be used. Thus, the stones can be substantially round, oval or oblong, or amorphous in shape. Geometric shapes, including faceted stones or crystals can also be used. Crystals are also known to have projections or protrusions, in the form of “fingers” or “arms” resulting from lateral growth or formation; such crystal formations are also useful in accordance with the subject invention. In addition, the chakra stones or crystals can be honed or shaped to provide any particular desired shape, using a mechanical grinder or by being “cut” such as with a stone or crystal cutter, e.g., a diamond cutter.

Sizes and shapes of chakra stones are well known by persons of ordinary skill in the art of chakra energies and the conventional sizes and shapes of chakra stones and crystals are intended to be encompassed by the subject invention. Sizes and shapes of chakra stones that are not currently used in conventional therapies or procedures, but are later developed and recognized as having particular beneficial energies, are also within the scope of the invention and can be useful components of the composition or article of manufacture described herein.

FIG. 2 is identical to FIG. 1, showing the article of manufacture 10 comprising a container 20 containing chakra stones 30A-30G within composition 40, but further illustrates written instruction or information 50 imprinted onto or attached to the container to provide instruction or information regarding the composition (such as a list of ingredients or manufacturing information), the chakra stone or stones (such as information regarding the color of the stone or its association with a chakra energy of the body), uses thereof (e.g., directions for use of a cleansing composition contained therein or instructions for realigning a chakra energy using a chakra stone or stones provided therein), or a combination of such information or instruction.

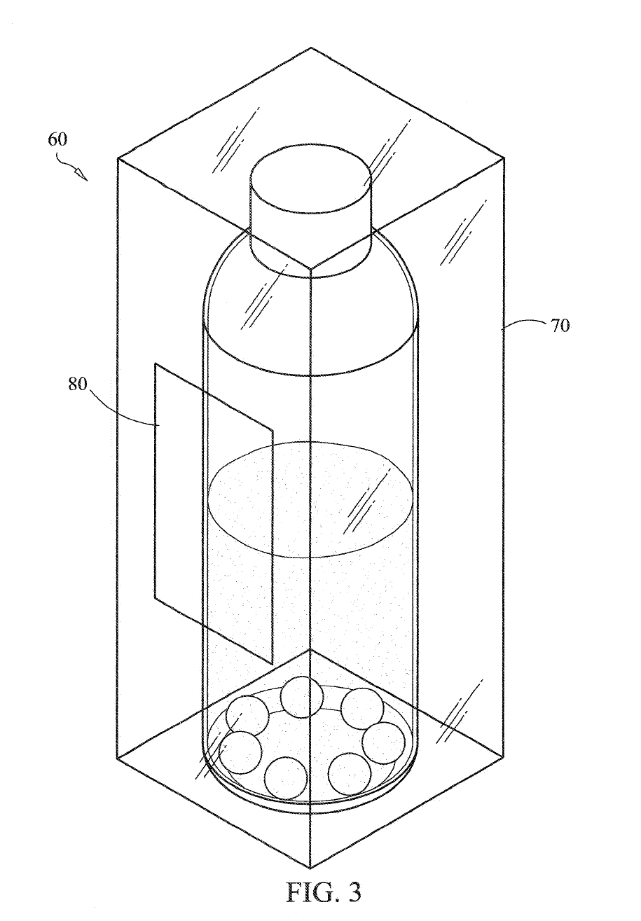

FIG. 3 also illustrates an article of manufacture 60 showing container 20 comprising chakra stones 30A-30G within composition 40, wherein the container is housed or packaged within packaging 70. Written instruction or information 80 (the same or different than 50 of FIG. 2) can also be imprinted onto or provide separately and attached to the packaging 70.

The seven power points in the body, referred to as chakras, circulate energy or the life force (known as “prana”). These chakras are vertically aligned in the center of the body, close to the spine. Unbalanced chakras can negatively affect one's physical, mental and/or spiritual health. Balancing unbalanced chakras, by realigning, revitalizing, or replenishing the energies of the body can be achieved by the use of a particular stone or crystal which provides a particular energy, vibration or frequency. By pulling the frequency of each stone's energy through the associated chakra of the body, the realignment or balancing can be effected.

Generally, the chakras are understood to represent or comprise the following:
1. CROWN CHAKRA—SEVENTH CHAKRA: Located above the head. This chakra links the individual with the universal.
2. THIRD EYE CHAKRA—SIXTH CHAKRA: Located between the eyes. This is intelligence and psychic power.
3. THROAT CHAKRA—FIFTH CHAKRA: Located at the throat. This is the creativity, self-expression and the search for truth.
4. HEART CHAKRA—FOURTH CHAKRA: Located at the heart. This is the higher consciousness and love.
5. SOLAR PLEXUS CHAKRA—THIRD CHAKRA: Located at the solar plexus. This is the sense of identity.
6. SACRAL HARA CHAKRA—SECOND CHAKRA: Located at the genitals. This is the chakra that monitors sexuality and reproduction.
7. ROOT CHAKRA—FIRST CHAKRA: Located at the base of the spine and groin area. This is the animal or base nature.

Although seven chakras are generally accepted, the human energy field may be made up of a plurality of minor chakras in addition to the major seven. Additional chakras, known as the “etheric chakras” include: the Soul Star Chakra, the Thymus or Higher Heart Chakra, and the Earth Chakra.

Although most chakra stones of a specific color will be the stone for affecting a particular chakra or chakra energy of the body, it is understood that some stones may be used to affect, realign, revitalize or balance more than one chakra. There are many stones for each chakra, and various crystals may be particularly powerful to aid healing or realigning an unbalanced chakra in different ways. There are a wide range of alignment attributes that pertain to various stones and the things that they are known to remedy.

Colors associated with each chakra of the body are provided in Table 1, below.

TABLE 1

| Chakra | Chakra Color |
|---|---|
| Soul Star Chakra | White |
| Crown Chakra | Violet and White |
| Third Eye Chakra | Purple |
| Throat Chakra | Blue |
| Thymus or Higher Heart Chakra | Pink and Turquoise |
| Heart Chakra | Green and Pink |
| Solar Plexus Chakra | Yellow |
| Navel or Sacral Chakra | Orange |
| Root or Base Chakra | Red and Black |
| Earth Star or Earth Chakra | Black and Brown |

Stones or crystals that are generally of a color, as listed above, are associated with realignment of that chakra. Some stones do not fit the pattern. For example, clear crystals, black, brown and various mixed color crystals can have a healing effect on more than one chakra. Other stones that heal more than one chakra are those with flecks of color through them, such as agates.

Clear quartz crystals are associated with realignment of all chakras of the body.

Red, associated with the Root or Base Chakra, symbolizes action, confidence, courage, vitality. Red Coral, Bloodstone, Garnet, Red Jasper, Ruby, and Red Calcite are stones or crystals that can be used in association with realignment of the Base Chakra.

The Base Chakra and Earth Chakra are also represented by black, which symbolizes death. Black Tourmaline, Obsidian, Onyx, Black Kyanite, Jet, Shungite, and Nuummite can be used in association with the realignment of the Base and Earth Chakras.

Pink, associated with the Heart or Higher Heart (Thymus) Chakra, symbolizes love and beauty. Rhodochrosite, Rhodonite, Rose Quartz, Pink Opal, Pink Tourmaline, Danburite, Pink Mangano, Calcite, Stilbite, Morganite, Chalcedony, Petalite, Pink Sapphire, Pink Halite, and Cobaltoan Calcite are stones or crystals that can be used in association with realignment of these chakras.

Brown, associated with the Earth Chakra, symbolizes earth, order, and convention. Tiger Eye, Petrified Wood, Dravite (brown tourmaline), Chiastolite, Aragonite, Stromatolite, Staurolite, Bronzite, Axinite, Moqui Ball, are stones or crystals that can be used in association with realignment of the Earth Chakra.

Orange, associated with the Navel or Sacral Chakra, symbolizes vitality and endurance. Carnelian, Fire Opal, Sunstone, Orange Calcite, Tangerine Quartz, Orange Kyanite, and Orange Elestial are stones or crystals that can be used in association with the realignment of the Navel or Sacral Chakra.

Yellow, associated with the Solar Plexus Chakra, symbolizes wisdom, joy, happiness, and intellectual energy. Amber, Citrine, Imperial Topaz, Lemon Quartz, Honey Calcite, Yellow Fluorite, and Sulphur are stones or crystals that can be used in association with the realignment of the Solar Plexus Chakra.

Green, associated with the Heart Chakra, symbolizes life, nature, fertility, and well-being. Emerald, Malachite, Aventurine, Jade, Peridot, Moss Agate, Chrysoprase, Serpentine, Green Calcite, Green Tourmaline, Dioptase, Prehnite, Infinite Stone, Tree Agate, Bloodstone, Seraphinite, Green Chlorite, Green Sardonyx, Green Irish Blessings stones, Fuchsite, Butterstone, Epidote, Prasiolite, Moldavite, and Hiddenite are stones or crystals that can be used in association with the realignment of the Heart Chakra.

Blue, which symbolizes youth, spirituality, truth, and peace and Indigo, which symbolizes intuition, meditation, deep contemplation are associated with the Throat Chakra. Blue stones, such as Turquoise, Aquamarine, Blue quartz, Lapis Lazuli, Blue lace agate, Blue Calcite, Blue Kyanite, Blue Apatite, Angelite, Dumortierite, Chrysocolla, Sapphire (mine rough), Blue Aventurine, Celestite, Blue Aragonite, Blue Tourmaline, Larimar, Hemimorphite, Sodalite, and Tanzanite, and indigo stones, such as indigo, sapphire, azurite, and lolite, are stones or crystals that can be used in association with realignment of the Throat Chakra.

Purple, which symbolizes royalty, magic, and mystery, is associated with the Third Eye Chakra. Amethyst, Fluorite, Sugilite, Lepidolite, Charoite and Purpurite can be used in association with realignment of the Third Eye Chakra.

White is associated with the Crown Chakra. White is also associated with the Star Soul Chakra. White symbolizes purity and cleanliness. White chakra stones include Crystal quartz, White Topaz, White Aragonite, Howlite, Scolecite, White Selenite, White Opal, Opalite, White Kunzite, and Ulexite can be used in association with realignment of the Crown and Star Soul Chakras. In some instances, violet, which symbolizes purpose, is also associated with the Crown Chakra. Accordingly, Amethyst, Fluorite, Sugilite, Lepidolite, Charoite and Purpurite can be used in association with realignment of the Crown Chakra.

Grey is a neutral color that symbolizes sorrow, security, and maturity. Hematite and Galena can be used for a neutral activity for a chakra.

Methods of using the composition of the subject invention include the conventional use of the cleansing composition to clean, condition, moisturize an area of the body such as the hair, nails, or skin. The method employs the steps of providing the composition or article of manufacture comprising the cleansing composition and at least one chakra stone or crystal, and periodically applying the composition to the area of the body intended to be cleansed, conditioned, or moisturized.

For example, a shampoo composition comprising at least one chakra stone or crystal can be used to wash a person's hair. For a person in need of realignment of a particular chakra of the body, the shampoo can be used to realign that chakra concomitantly with carrying out the cleansing purpose of the shampoo. A shampoo composition comprising one or more chakra violet or white chakra stones or crystals can be used to wash the hair of a person in need of realignment of, for example, the Crown Chakra. This shampoo composition can be used periodically or routinely, e.g., daily or 2-3 times per week, or as needed.

Alternatively, the composition can comprise a plurality of different colored chakra stones or crystals for affecting or realigning a plurality of chakras of the body. A shampoo comprising at least seven different colored chakra stones or crystals (e.g., in accordance with the color scheme of Table 1) associated with each of the seven major chakras of the body can be used to affect or realign one or more of those seven major chakras of the body.

Other methods of using a composition or article of manufacture of the subject invention include using the chakra stones or crystals separate from the cleansing composition. For example, the chakra stones can be removed from the container and used in a routine, therapy or procedure to realign or balance an unbalanced chakra of the body. The one or more chakra stones or crystals can be removed prior to or after the cleansing composition is used.

Alternatively, the chakra stone or crystal can be removed from the container to create a mandala. “Mandala” is a Sanskrit word meaning “circle” and has religious significance in the Hindu and Buddhist religions. In various spiritual traditions, mandalas may be employed as a spiritual teaching tool, for establishing a sacred space, and as an aid to meditation and trance induction. In common use, mandala has become a generic term for any plan, chart or geometric pattern that represents the cosmos metaphysically or symbolically, a microcosm of the universe from the human perspective.

A method of creating a mandala using the chakra stones or crystals of the subject invention comprises the steps of:
1. remove the one or more chakra stones or crystals from the container containing or formerly containing the cleansing composition;
2. create a quiet, undisturbed space in the home or office;
3. Using the chakra stones or crystals, which can optionally be cleaned or rinsed prior to their use, put a clear chakra stone or crystal, such as a crystal quartz or diamond, in the center of a space and surround the clear chakra stone or crystal with a chakra stone or crystal having a color associated with each of the seven chakras of the body; and
4. Physically or emotionally placing oneself in the center of the mandala to absorb the energy or frequencies given from the mandala of chakra stones or crystals.

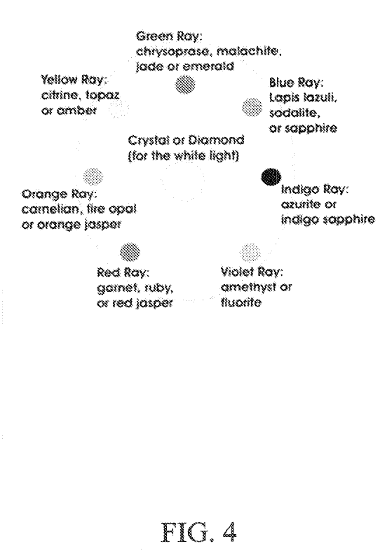

An example of a mandala arrangement of chakra stones or crystals according to a method of the invention is shown in FIG. 4.

The method of creating a mandala using the chakra stones or crystals from the cleansing composition of the subject invention can further comprise adding an altar that can comprise fresh flowers, a personal totem, a scented candle, or the like.

The foregoing description of the invention has been presented for purposes of illustration to enable any person skilled in the art to which the invention pertains to make and use it. The best mode contemplated by the inventor of carrying out this invention is not intended to be exhaustive or to limit the invention to the precise form disclosed. The teachings of the description can be used to make other modifications and variations. The embodiment was chosen and described to explain the principles of the invention and its practical application, thereby enabling those skilled in the art to which the invention pertains to utilize the invention in various embodiments and modifications suited to the particular use contemplated.

1. A personal hygiene composition for applying to an area of a body, said composition comprising a cleansing, conditioning, hydrating, moisturizing, or cosmetic composition and further comprising at least seven chakra stones or crystals, wherein each of the seven chakra stones or crystals is associated with at least one of at least seven different chakras or chakra energies of the body and wherein said cleansing, conditioning, hydrating, moisturizing, or cosmetic composition is a transparent or translucent liquid or gel selected from the group consisting of soap, shampoo, and hair conditioner.

2. The composition of claim 1 wherein the cleansing, hydrating, moisturizing, or cosmetic composition is a shampoo.

3.-5. (canceled)

6. An article of manufacture comprising a personal hygiene composition of claim 1 and a container for holding or containing the composition.

7. The article of manufacture of claim 6, said article further comprising a written instruction or written information regarding the composition and the use thereof.

8. (canceled)

9. The article of manufacture of claim 6 wherein the container is selected from the group consisting of a bottle, a dispensing tube, a tub, a jar, and a canister.

10. The article of manufacture of claim 6 wherein the container comprises a transparent or translucent material allowing visibility of the composition and the at least one chakra stone or crystal therein.

11. The article of manufacture of claim 6, wherein the article of manufacture is a novelty item.

12. The article of manufacture of claim 6, wherein the article of manufacture is a decorative item.

13. The article of manufacture of claim 6 further comprising an outer packaging, said outer packaging housing the container holding the cleansing, conditioning, hydrating, moisturizing, or cosmetic composition and the at least one chakra stone or crystal.

14. The article of manufacture of claim 13 wherein the outer packaging comprises a written instruction or written information regarding the cleansing, conditioning, hydrating, moisturizing, or cosmetic composition, the at least one chakra stone or crystal, or the use thereof.

15. A method for using a composition of claim 1, said method comprising
providing the composition of claim 1 comprising the cleansing, conditioning, hydrating, moisturizing, or cosmetic composition and at least one chakra stone or crystal, and
applying the cleansing, conditioning, hydrating, moisturizing, or cosmetic composition to an area of the body intended to be cleansed, conditioned, or moisturized.

16. The method of claim 15 wherein the method further comprises concomitantly realigning or balancing a chakra of the body by applying the cleansing, conditioning, hydrating, moisturizing, or cosmetic composition comprising at least one chakra stone or crystal to the body.

17. The method of claim 16 wherein the composition comprises at least one chakra stone or crystal representing each of seven chakras of the body.

18. A method for using a composition of claim 1, said method comprising
removing the one or more chakra stones or crystals from the container, and
realigning or balancing an unbalanced chakra of the body by performing a traditional chakra realigning treatment, therapy or procedure.

19. The method of claim 18, wherein the composition comprises at least one chakra stone representing each of the seven chakras of the body, the method further comprising forming or arranging a mandala from the chakra stones or crystals of the composition, said mandala comprising a centrally positioned clear chakra stone or crystal.
